(19) United States
(12) Patent Application Publication
(10) Pub. No.: US 2011/0098797 A1
(43) Pub. Date: Apr. 28, 2011

(54) DRUG ELUTING COMPOSITE

(76) Inventors: Robert L. Cleek, Flagstaff, AZ (US); Edward H. Cully, Flagstaff, AZ (US); Theresa A. Holland, Flagstaff, AZ (US)

(21) Appl. No.: 12/909,609

(22) Filed: Oct. 21, 2010

Related U.S. Application Data
(60) Provisional application No. 61/254,643, filed on Oct. 23, 2009.
(60) Provisional application No. 61/259,491, filed on Nov. 9, 2009.

(51) Int. Cl. A61K 31/573; A61K 9/00; A61P 29/00; A61N 1/05

(52) U.S. Cl. 607/120; 514/180; 424/422

(57) ABSTRACT

The present invention relates to materials having therapeutic compositions releasably contained within the materials. The materials are configured to release therapeutic compositions at a desired rate. The present invention also relates to devices incorporating the materials.

### CROSS-REFERENCE TO RELATED APPLICATIONS

This application claims priority to provisional application U.S. Ser. No. 61/254,643, filed Oct. 23, 2009, and provisional application U.S. Ser. No. 61/259,491 filed Nov. 9, 2009.

### FIELD OF THE INVENTION

The present invention relates to medical devices.

### SUMMARY OF THE INVENTION

The present invention relates to materials capable of releasing a therapeutic agent contained within the invention at determined concentrations for determined periods of time. Pathways are present within the material of the invention for therapeutic agents to traverse. The pathways extend the distance therapeutic agents contained within the invention must travel to exit the invention. The time taken for therapeutic agents to exit the invention is also extended by the pathways. Pathways are established in the present invention with combinations of permeable and impermeable compositions and/or structures located within the material containing the therapeutic agents. Compositions and/or structures impermeable to a selected therapeutic agent are also used as barriers to the therapeutic agent on one or more surfaces of the invention. As a result, the therapeutic agent can only exit the invention in areas not covered, contacted, or otherwise constructed with compositions and/or structures impermeable to the selected therapeutic agent. Openings are also provided in the compositions and/or structures impermeable to a selected therapeutic agent in some embodiments of the invention.

Embodiments of the present invention can be used alone or in combination with other embodiments of the invention. The invention can also be a component of a device such as cardiac pacing devices, cardiac defibrillation devices, neurostimulation devices, endoprosthesis such as grafts and stent-grafts, interventional devices such as catheters and filters, diagnostic devices such as transducers, sensors, and other medical devices placed in contact with living tissue responsive to one or more therapeutic agents.

Implantable embodiments of the invention can be used to elute an anti thrombogenic drug into a left atrial appendage. Prevention of blood clots in this anatomical region could obviate the need for a left atrial appendage occluder. In this embodiment, the therapeutic composition, agent, or compound could be high in concentration when implanted and rapidly diluted when the blood is washed out into the heart and circulatory system.

Accordingly, one embodiment of the present invention relates to a therapeutic-releasing material comprising a first biocompatible polymeric material having at least one surface and a therapeutic agent releasably incorporated in at least a portion thereof, wherein a portion of said first biocompatible polymeric material is impermeable to said therapeutic agent, and a second biocompatible polymeric material impermeable to said therapeutic agent covering substantially all said at least one surface.

Another embodiment of the present invention relates to a therapeutic-releasing material comprising a porous biocompatible polymeric material having at least one surface, a therapeutic agent releasably admixed with a biocompatible fluoropolymeric copolymer and incorporated in pores of said porous biocompatible polymeric material, wherein a portion of said porous biocompatible polymeric material is impermeable to said therapeutic agent, and a nonporous biocompatible polymeric material impermeable to said therapeutic agent covering substantially all said at least one surface.

A further embodiment of the present invention relates to a first biocompatible polymeric material in the form of a film having at least one surface and a therapeutic agent releasably incorporated in at least a portion of said film, wherein a portion of said first biocompatible polymeric material is impermeable to said therapeutic agent, and a second biocompatible polymeric material impermeable to said therapeutic agent covering substantially all said at least one surface of said film.

Other embodiments of the present invention relate to medical devices having a therapeutic-releasing material incorporated therein. For example, one embodiment relates to a cardiac pacing or Intracardiac Cardioverter Defibrillation (ICD) leads comprising a cardiac lead element having a proximal end and a distal end, an electrically conductive connector at said proximal end, an electrode located at said distal end, at least one electrically conductive element connecting said connector to said electrode, and at least a portion of said cardiac element covered with a therapeutic-releasing material having a first biocompatible polymeric material having at least one surface and a therapeutic agent releasably incorporated in at least a portion thereof, wherein a portion of said first biocompatible polymeric material is impermeable to said therapeutic agent and a second biocompatible polymeric material impermeable to said therapeutic agent covering substantially all said at least one surface.

Another embodiment relates to an electrically conductive lead comprising a lead element having a proximal end and a distal end, an electrically conductive connector at said proximal end, an electrode located at said distal end, at least one electrically conductive element connecting said connector to said electrode, a tubular lead tip located at said distal end, and at least a portion of said lead element covered with a therapeutic-releasing material having a first biocompatible polymeric material having at least one surface and a therapeutic agent releasably incorporated in at least a portion thereof, wherein a portion of said first biocompatible polymeric material is impermeable to said therapeutic agent and a second biocompatible polymeric material impermeable to said therapeutic agent covering substantially all said at least one surface.

In each embodiment of the present invention, at least one opening can be placed in the impermeable materials and impermeable portions of the invention to provide a path for therapeutic agents to be released from, or otherwise travel through, the material.

### BRIEF DESCRIPTION OF THE DRAWINGS

FIG. 1 illustrates a perspective view of an embodiment the present invention.

FIG. 1A illustrates a transverse cross section taken at line “C” in FIG. 1.

FIG. 2 illustrates a perspective view of another embodiment of the present invention.

FIG. 2A illustrates a transverse cross section taken at line “D” in FIG. 2.

FIG. 3 illustrates a perspective view of the embodiment of FIG. 2.

FIG. 4 is a graph.

FIG. 5 illustrates an embodiment of the present invention.

FIG. 6 illustrates a means for delivery or retrieval of the invention.

FIG. 7 illustrates an embodiment of the present invention.

### DETAILED DESCRIPTION OF THE INVENTION

The present invention relates to materials having therapeutic compositions releasably contained within the materials. The materials are configured to release therapeutic compositions at a desired rate. The present invention also relates to devices incorporating the materials. In preferred embodiments, materials and/or constructions bar, or otherwise impede, movement of therapeutic compositions present within the material of the invention. Some embodiments have materials and/or constructions reducing, or otherwise limiting, the rate of release of therapeutic compositions from the invention, but not barring, blocking, or otherwise impeding movement of a therapeutic composition through the invention.

The rate at which therapeutic agents are released from the invention is influenced by several factors. These include the chemical composition of the components of the invention, the physical relationship of the components, the overall shape of the invention, and any openings provided in the invention. The chemical composition of the components of the invention include formulations of the therapeutic agent and materials containing the therapeutic agent, such as mass fractions, presence or absence of expedients, and the magnitude of the diffusion coefficient for the invention.

Combinations of compositions and/or structures permeable to therapeutic agents and compositions and/or structures impermeable to therapeutic agents are used in the present invention to establish a pathway along which therapeutic agents move as the agents move through and out of the invention. As a result, therapeutic agents are preferentially eluted, or otherwise released, from permeable portions of the material and not impermeable portions.

A notable advantage of the invention is the ability to control the release rate concurrently with the total percentage of therapeutic compositions released. Some therapeutic compositions are unstable and it is not desirable to leave large or even small portions of the compositions remaining within the invention for periods of time. With more traditional approaches, the rate of release is controlled through the mixture of the therapeutic compositions and a polymer. Unlike the present invention, therapeutic compositions can remain within a conventional device permanently or for undesirable periods of time.

In addition, the invention has a variety of configurations which can influence the rate at which therapeutic agents are released from the invention. The configurations include films, sheets, rods, tubular shapes having luminal spaces, hollow or solid spherical shapes, laminates, wraps, and other shapes.

The material of the present invention includes therapeutic compositions, agents, or compounds such as small molecule drugs, large molecule drugs, medicaments, cardiovascular agents, chemotherapeutics, antimicrobials, antibiotics, anesthetics, hemostatics, antihistamines, antitumors, antilipids, antifungals, antimycotics, antipyretics, vasodilators, hypertensive agents, oxygen free radical scavengers, antivirals, analgesics, antiproliferatives, antiinflammatories, diagnostic agents, visualization agents, angiographic contrast agents, phase contrast agents, and radiopaque agents, or thrombolytics intended to facilitate the breakup of thrombus, anticoagulants such as heparin, intended to prevent thrombosis and combinations thereof. The therapeutic composition may be an anti-inflammatory steroid such as dexamethasone sodium phosphate, dexamethasone acetate, dexamethasone, and/or beclomethasone dipropionate.

Yet other therapeutic compositions include, but are not limited to, antirestenotic drugs including, but not limited, to pimecrolimus, cytochalasin, dicumarol, cyclosporine, latrunculin A, methotrexate, tacrolimus, halofuginone, mycophenolic acid, genistein, batimistat, dexamethasone, cudraflavone, simvastatin, prednisolone, doxorubicin, bromopyruvic acid, carvedilol, mitoxantrone, tranilast, etoposide, hirudin, trapidil, mitomycin C, abciximab, cilostazol, irinotecan, estradiol, diaziquone, dipyridamole, melatonin, colchicine, nifedipine, vitamin E, paclitaxol, diltiazem, vinblastine, verapamil, vincristine, rapamycin, angiopeptin, everolimus, heat shock proteins, zotarolimus, nitroglycerin, and prednisone.

In a preferred embodiment of the present invention, a film material permeable to a therapeutic compound is impregnated or coated with a copolymer into which has been admixed the therapeutic compound. The preferred film material is an expanded polytetrafluoroethylene (ePTFE) construction. The copolymer is preferably a tetrafluoroethylene/perfluoromethylvinylether (TFE/PMVE) copolymer.

A material impermeable to the therapeutic composition, agent, or compound is placed on at least one surface of the therapeutic-containing film material to prevent movement of the therapeutic agent or compound through or out of the invention at the location of the impermeable material. In some embodiments, the impermeable material has at least one opening therein. The material for the “capping layer” is preferably formed of a polymer such as a silicone composition. Depending on the embodiment, the capping layer material is applied either to a portion of the coated film material or all of the film material. The portion of the coated film material which is not covered by the capping layer material preferentially elutes the therapeutic composition, agent, or compound when exposed to fluids. The capping layer material may be applied over the coated film material after the film material is applied to a substrate.

In some embodiments, the present invention is combined with a substrate in the form of a device or other construction. In these embodiments, a coated film material is applied to all or a portion of the substrate underlying the invention. The coated film material may be cut into a tape and applied by wrapping the tape around the substrate. The tape is wrapped helically and/or longitudinally around at least a portion of the substrate. The coated film may be applied to the substrate with the coated side facing the substrate or facing away from the substrate. Substrates may include tubes, rods, pellets, or any other three dimensional object, including substrates which may be a component of an assembled device. Substrates may be made of metals, polymers, and the like. The substrate may be shaped or altered to form elution pathways through and out of the present invention,

As used herein, the term “bioabsorbable” refers to a physiological process in which at least a portion of a material hydrolyzes, degrades, or otherwise dissolves in living tissue or biological fluid.

As used herein, the term “permanent implant” refers to a medical device intended to be implanted in a patient for all or most of the life of the patient.

As used herein, the term “semi-permanent implant” refers to a medical device intended to be implanted in a patient for less than the expected life of the patient.

Semi-permanent implants are often accessed following implantation for removal of the device or other procedure related to the device.

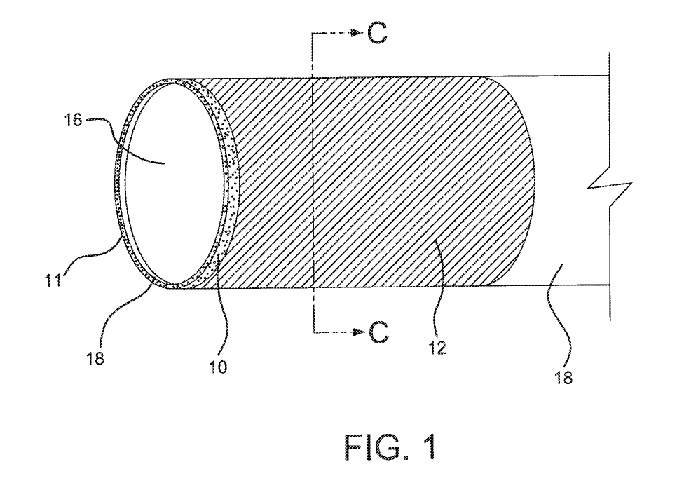

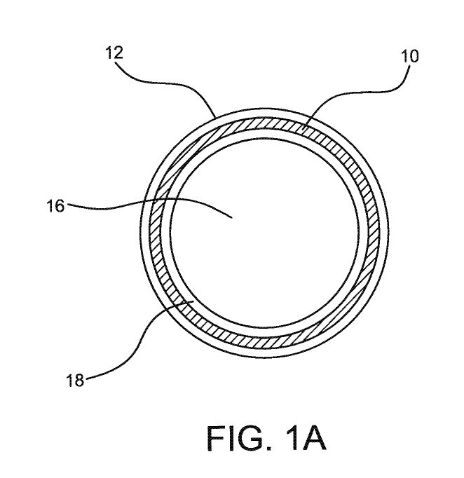

Referring to FIG. 1, coated film (10) has a therapeutic composition, agent, or compound (not shown) incorporated into the film. Coated film (10) is applied over a substrate (18). A capping layer (12) is applied over coated film (10). The capping layer (12) is either made of materials impermeable to the particular therapeutic composition, agent, or compound or constructed to be impermeable to the particular therapeutic composition, agent or compound.

In this embodiment, the substrate (18) is a tubular structure with a luminal space (16). Material of the capping layer (12) covers only a portion of the coated film material (10) thereby leaving a portion of coated film material exposed around an edge, or lip, of the substrate (18). The exposed portion of the coated film material (10) has a thickness dimension (11).

This embodiment is also illustrated in FIG. 1A as a transverse cross section taken at line “C” in FIG. 1 showing substrate material (18), luminal space (16), coated film material (10) and capping layer material (12).

In practice, the embodiment illustrated in FIG. 1 is placed in contact with a bodily tissue or fluid. Once in contact with tissue and/or fluid, the therapeutic composition, agent, or compound (not shown) contained within coated film (10) is preferentially eluted from those portions of the coated film material not covered by material of the capping layer (12). In this embodiment, for example, the therapeutic composition, agent, or compound elutes or otherwise exits the invention from an uncapped, or otherwise uncovered, edge (11) surrounding the opening of luminal space (16). The therapeutic composition, agent, or compound in the coated film material (10) may diffuse, or otherwise migrate, from portions of the coated film material (10) covered by material of the capping layer (12) and exit the invention from uncovered and exposed areas of the coated film material (10).

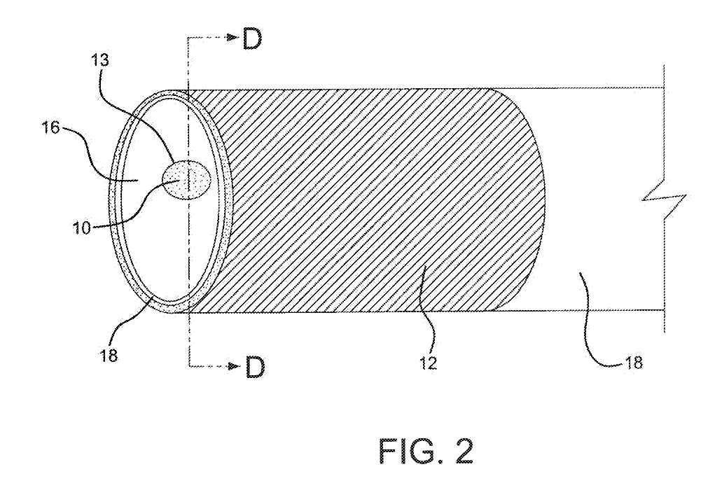

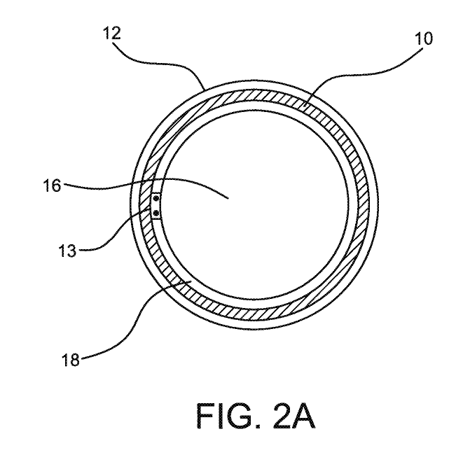

Another embodiment of the present invention is illustrated in FIG. 2. In this embodiment, coated film material (10) has a therapeutic composition, agent, or compound (not shown) incorporated into the film. The coated film material (10) is applied over a substrate (18). A capping layer material (12) is applied over the entire exterior surface of coated film material (10). The capping layer (12) is either made of materials impermeable to the particular therapeutic composition, agent, or compound or constructed to be impermeable to the particular therapeutic composition, agent, or compound. An opening (13) in the form of a hole is made through substrate 18, exposing coated film material (10) to the luminal space (16) of the substrate (18). A porous material may be placed over opening (13) and between the substrate (18) and coated film material (10). Additionally, this material placed over opening (13) may modulate release of a therapeutic composition, agent, or compound.

FIG. 2A is a transverse cross section taken at line “D” in FIG. 2 showing substrate (18), luminal space (16), coated film material (10), capping layer material (12), and opening (13).

In practice, the embodiment illustrated in FIG. 2 is placed in contact with a tissue or fluid. Once in contact with tissue and/or fluid, the therapeutic composition, agent, or compound in coated film material (10) preferentially elutes through opening (13) and out of luminal space (16) into surrounding fluid and/or tissues (not shown). The therapeutic composition, agent, or compound in coated film material (10) may migrate to opening (13) from portions of coated film material (10) covered by capping layer material (12) and located away from opening (13).

FIG. 3 is a perspective view of the embodiment illustrated in FIG. 2 except cover material (17) covers luminal space (16) as shown in FIG. 2. Optionally, an opening (20) can be made in cover material (17) through which tissue fixation means (19), such as a screw may be included. Additional means of tissue fixation include appropriate anchors, barbs, hooks or adhesives. The tissue fixation means can be made of metallic or polymeric materials. The metallic or polymeric materials can be bioabsorbable or non-bioabsorbable. An example of a bioabsorbable metal is magnesium. An example of a bioabsorbable polymer is polyglycolic acid commonly known as PGA.

In practice, the embodiment illustrated in FIG. 3 is anchored into tissue using tissue fixation screw (19) and the therapeutic composition, agent, or compound in coated film material (10) is allowed to preferentially elute from opening (13) into luminal space (16) and out of opening (20) into surrounding tissues and/or fluids. The embodiment illustrated in FIG. 3 may be used for implantation into the heart and other tissues as described below. For example, in cardiac leads a tissue fixation screw (19) is often placed into the septum of the right ventricle.

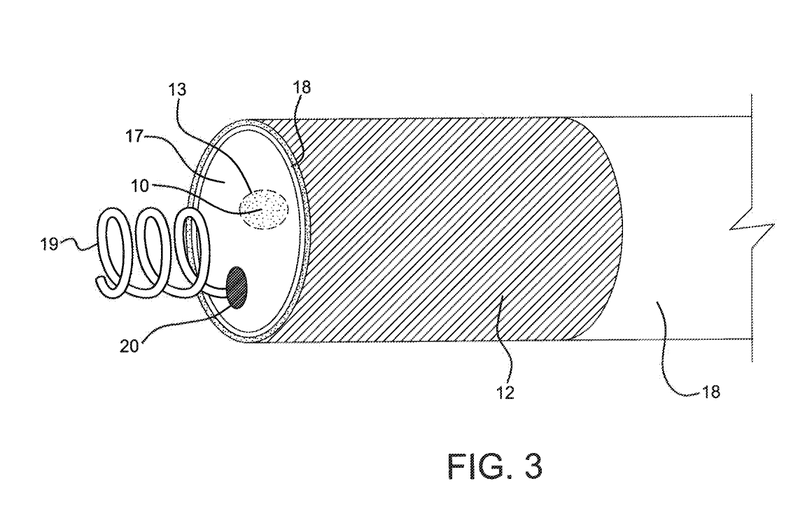

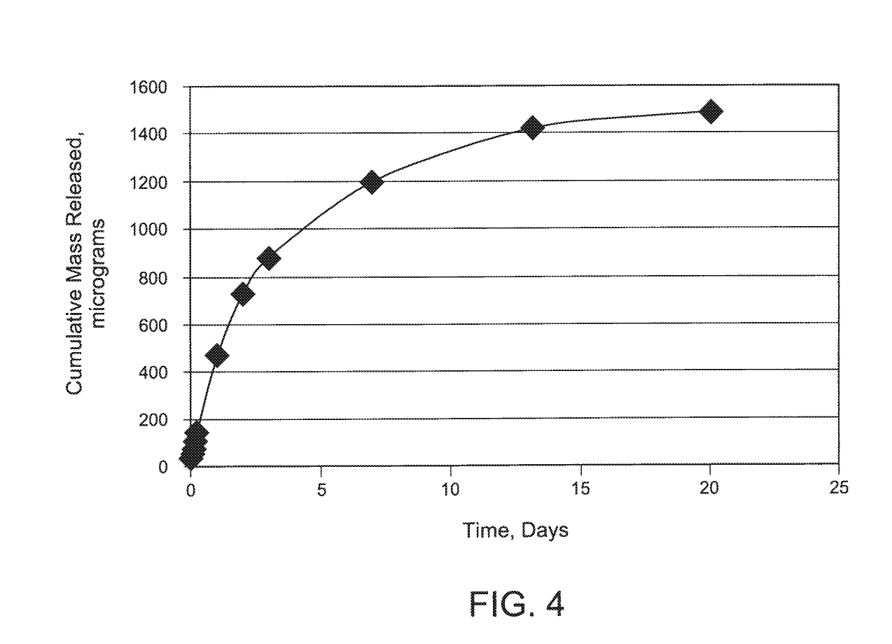

FIG. 4 is a graph of the cumulative mass of drug released as a function of time for the embodiment described in Example 1.

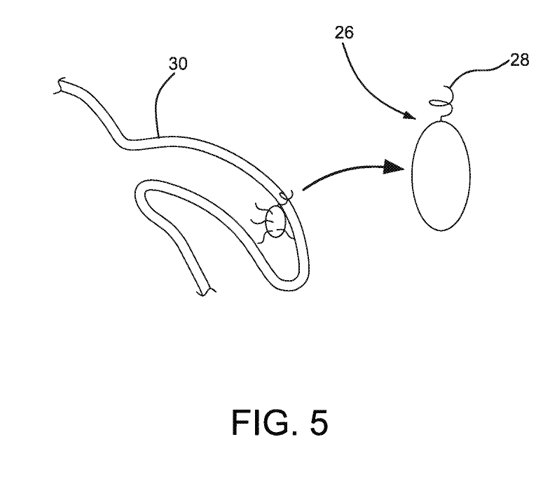

FIG. 5 illustrates another embodiment of the present invention. A housing (26) includes a therapeutic eluting construction of the present invention with a means to attach the housing (26) to tissue such as a tissue attachment screw (28). Depending on the application, the housing (26) may be attached to a tissue region or anatomical location such as a left atrial appendage (30). The attachment may be permanent or semi-permanent in the event the housing (26) is subsequently removed and optionally exchanged.

The housing (26) may be incorporated in the embodiment described in Example 1. The housing (26) may be made of metallic or polymeric materials. The housing (26) is solid, hollow, or include features such as perforations (32) as illustrated in FIG. 7.

In one embodiment, both a housing (26) and tissue attachment screw (28) are made of materials which are bioabsorbable. In one embodiment, the entire housing (26) is a solid bioabsorbable material having with a therapeutic composition, agent, or compound incorporated therein. Over time, the entire housing implant will hydrolyze, or otherwise dissolve, while eluting the therapeutic agent. In yet another embodiment, the therapeutic composition, agent, or compound incorporated within the bioabsorbable material may vary in both composition and concentration. For example, the housing (26) may be constructed such that the initial eluted dosage of therapeutic composition, agent, or compound may be very high, with potency dropping off over time as a function of variable bioabsorption produced by using materials of varying bioabsorbability. In one embodiment, such variable elution may be utilized by constructing a housing (26) with multiple layers of therapeutic-loaded bioabsorbable materials, each layer having a different therapeutic concentration or each layer having a different rate of bioabsorbability or a combination of both.

Elution rates may also be varied by modifying the housing (26). For example, the housing (26) may include perforations (32) as illustrated in FIG. 7. The perforations (32) permit elution from the inner regions of the housing (26) or increase surface area of the housing (26). In one embodiment, elution rates may be controlled by overwrapping or encasing a housing (26) within a porous or semi-permeable covering material (34) as illustrated in FIG. 7. A porous expanded polytetrafluoroethylene material exhibits is biocompatible and with substantial chemical inertness. A porous expanded polytetrafluoroethylene material for the overwrapping or encasing material is a preferred material.

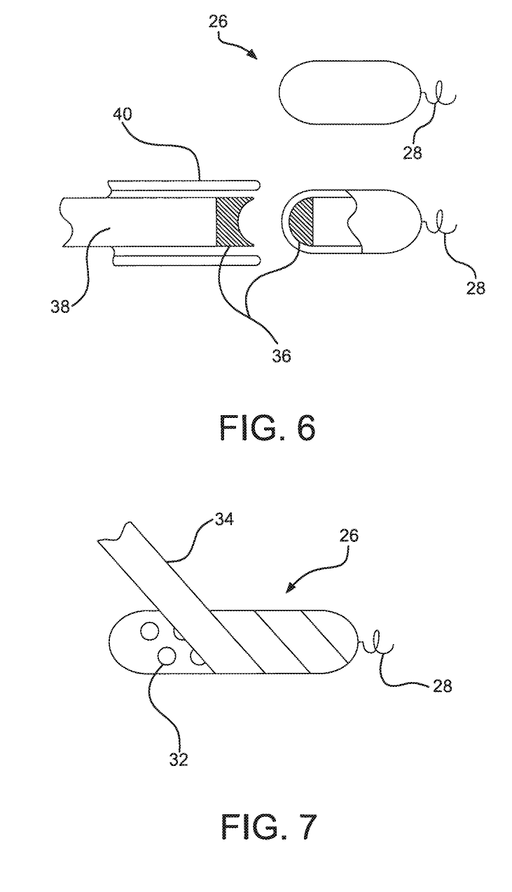

In some situations, it may be necessary to retrieve or replace an implanted embodiment of the present invention. Retrieval can be accomplished with a grasping tool. In one embodiment, magnetic attachment is used to retrieve or replace an implanted device (see e.g., FIG. 6). Magnets (36) may be embedded within or on the surface of the housing (26) and the associated catheter (38). The magnets (36) are configured exert an attractive force between the magnets. Once a sufficient magnetic attracting has been established, in-situ capture and movement of housing (26) can be effected. A sheath (40) may be used in the present invention. The sheath (40) is advanced over a housing (26) and the entire system rotated to cause release of the tissue attachment screw (28) and removal from the implant site.

Embodiments of the present invention may be configured for a variety of purposes, including therapeutic-eluting tips for cardiac pacing or Intracardiac Cardioverter Defibrillation (ICD), or neurostimulation leads; or other therapeutic-eluting devices for placement in proximity to other body tissues. Once placed at the desired location by interventional or surgical means and enclosed by tissue or affixed to tissue with an anchor, the invention can be of therapeutic value by locally or systemically delivering a drug. Although the left atrial appendage (30) implantation site is described herein, it should be appreciated the present invention may be applicable to a variety of other applications, such as the liver, kidney, brain, or peripheral vascular system. Accordingly, use of the present invention need not be constrained to the cardiovascular system. For instance, embodiments for implantation within a sinus cavity and loaded with an antihistamine or other allergy-symptom relieving agent are contemplated.

### EXAMPLES

### Example 1

A copolymer of tetrafluoroethylene/perfluoromethylvinylether (TFE/PMVE) as described in EP 1545642 B1 was obtained in a 0.12 wt % solution of Fluorinert FC-77 (3M, St Paul, Minn.). To this solution was added an appropriate amount of dexamethasone sodium phosphate (Pharmacia & Upjohn Company, Kalamazoo Mich.) to produce a solution of 0.12 wt % of the drug. The solution was sonicated to ensure complete mixing.

An expanded polytetrafluoroethylene (ePTFE) film tape of approximately 0.01 mm in thickness and 0.8 cm width was utilized in the manufacturing of the drug release system. A length of ePTFE film tape approximately 8 cm long was mounted onto a flat sheet of aluminum foil with a section of adhesive tape at each end. The ePTFE film tape was spray-coated with the TFE/PMVE and dexamethasone sodium phosphate solution using an airbrush (Badger standard set, model 350 (Badger Air Brush Co., Franklin Park, Ill.) set at 220 KPa gauge air pressure. Spray coating was conducted for 2-3 minutes, the coating was allowed to air dry, and the coated film then coated again. This was continued until the coating mass added to the tape was approximately 1 mg per 1 cm length. The opposite side of the film tape was left uncoated.

A metal tube of outside diameter of 1.50 mm, length 3 cm was obtained. A thin layer of a substantially non-porous composite film including expanded polytetrafluoroethylene (ePTFE) with a thermal adhesive layer of ethylene fluoroethylene perfluoride on one side was applied to the tube extending approximately 0.8 cm back from the tip of one end. This construct was utilized as a model cardiac pacing lead tip. The end of a segment of the coated film tape of 0.8 cm width and 2 cm in length was attached to the outer circumference of the tube, with the drug coated side facing the tube, at its end utilizing a silicone adhesive (MED-137, NuSil Technology, Carpinteria Calif.) and allowed to fully cure. After curing, a spatula was used to spread a thin film of the silicone adhesive on the coated side of the coated tape, and the tape was wrapped with the coated side toward the tube. The wrapped coated tape was then capped on a portion of its outer surface using silicone applied with a spatula, while not coating a thin strip of approximately 1 mm or less in width adjacent to the opening of the coated tape wrapped metal tube. The construct was allowed to cure overnight.

Constructs so made possessed a theoretical drug loading of approximately 2 mg and were tested for determination of drug release. A construct was placed in a vial containing 3 ml of PBS and maintained in a 37 degree C. incubator. Samples of 3 ml were taken at various time points and the vial replenished with fresh PBS to maintain the volume at 3 ml. Drug concentration was measured on an UV spectrophotometer at 242 nm. The graph shown in FIG. 4 demonstrates an extended elution time for the drug dexamethasone sodium phosphate.

1. A therapeutic-releasing material comprising:
a first biocompatible polymeric material having at least one surface and a therapeutic agent releasably incorporated in at least a portion thereof;
wherein a portion of said first biocompatible polymeric material is impermeable to said therapeutic agent; and
a second biocompatible polymeric material impermeable to said therapeutic agent covering substantially all said at least one surface.

2. The therapeutic-releasing material of claim 1 further comprising at least one opening in said second biocompatible polymeric material.

3. The therapeutic-releasing material of claim 1 further comprising a substrate material underlying said therapeutic-releasing material.

4. The therapeutic-releasing material of claim 2 further comprising a substrate material underlying said therapeutic-releasing material.

5. The therapeutic-releasing material of claim 1 wherein said first biocompatible polymeric material comprises a fluoropolymer composition.

6. The therapeutic-releasing material of claim 5 wherein said fluoropolymer is porous polytetrafluoroethylene.

7. The therapeutic-releasing material of claim 1 wherein said second biocompatible polymeric material is a silicone composition.

8. The therapeutic-releasing material of claim 1 wherein said therapeutic agent is dexamethasone sodium phosphate.

9. A therapeutic-releasing material comprising:
a porous biocompatible polymeric material having at least one surface;
a therapeutic agent releasably admixed with a biocompatible fluoropolymeric copolymer and incorporated in pores of said porous biocompatible polymeric material;
wherein a portion of said porous biocompatible polymeric material is impermeable to said therapeutic agent; and
a non-porous biocompatible polymeric material impermeable to said therapeutic agent covering substantially all said at least one surface.

10. The therapeutic-releasing material of claim 9 further comprising at least one opening in said non-porous biocompatible polymeric material.

11. The therapeutic-releasing material of claim 9 further comprising a substrate material underlying said therapeutic-releasing material.

12. The therapeutic-releasing material of claim 10 further comprising a substrate material underlying said therapeutic-releasing material.

13. The therapeutic-releasing material of claim 9 wherein said porous biocompatible polymeric material comprises a fluoropolymer composition.

14. The therapeutic-releasing material of claim 13 wherein said porous fluoropolymer is porous polytetrafluoroethylene.

15. The therapeutic-releasing material of claim 9 wherein said non-porous biocompatible polymeric material is a silicone composition.

16. The therapeutic-releasing material of claim 9 wherein said therapeutic agent is dexamethasone sodium phosphate.

17. A therapeutic-releasing material comprising:
a first biocompatible polymeric material in the form of a film having at least one surface and a therapeutic agent releasably incorporated in at least a portion of said film;
wherein a portion of said first biocompatible polymeric material is impermeable to said therapeutic agent; and
a second biocompatible polymeric material impermeable to said therapeutic agent covering substantially all said at least one surface of said film.

18. The therapeutic-releasing material of claim 17 further comprising at least one opening in said second biocompatible polymeric material.

19. The therapeutic-releasing material of claim 17 further comprising a substrate material underlying said therapeutic-releasing material.

20. The therapeutic-releasing material of claim 19 wherein said substrate is at least a portion of a cardiac pacing lead wire.

21. The therapeutic-releasing material of claim 19 wherein said therapeutic-releasing material is wrapped at least once around said substrate material.

22. The therapeutic-releasing material of claim 21 wherein said substrate is at least a portion of a cardiac pacing lead wire.

23. The therapeutic-releasing material of claim 21 wherein said wrapped therapeutic-releasing material has a spiral configuration.

24. The therapeutic-releasing material of claim 23 wherein said substrate is at least a portion of a cardiac pacing lead wire.

25. The therapeutic-releasing material of claim 17 wherein said first biocompatible polymeric material comprises a fluoropolymer composition.

26. The therapeutic-releasing material of claim 25 wherein said fluoropolymer is porous polytetrafluoroethylene.

27. The therapeutic-releasing material of claim 17 wherein said second biocompatible polymeric material is a silicone composition.

28. The therapeutic-releasing material of claim 17 wherein said therapeutic agent is dexamethasone sodium phosphate.

29. A cardiac pacing leading comprising:
a cardiac pacing lead element having a proximal end and a distal end;
an electrically conductive connector at said proximal end;
an electrode located at said distal end;
at least one electrically conductive element connecting said connector to said electrode; and
at least a portion of said cardiac pacing element covered with a therapeutic-releasing material having a first biocompatible polymeric material having at least one surface and a therapeutic agent releasably incorporated in at least a portion thereof, wherein a portion of said first biocompatible polymeric material is impermeable to said therapeutic agent and a second biocompatible polymeric material impermeable to said therapeutic agent covering substantially all said at least one surface.

30. The therapeutic-releasing material of claim 29 further comprising at least one opening in said second biocompatible polymeric material.

31. The therapeutic-releasing material of claim 29 wherein said first biocompatible polymeric material comprises a fluoropolymer composition.

32. The therapeutic-releasing material of claim 31 wherein said fluoropolymer is porous polytetrafluoroethylene.

33. The therapeutic-releasing material of claim 29 wherein said second biocompatible polymeric material is a silicone composition.

34. The therapeutic-releasing material of claim 29 wherein said therapeutic agent is dexamethasone sodium phosphate.

35. An electrically conductive lead comprising:
a lead element having a proximal end and a distal end;
an electrically conductive connector at said proximal end;
an electrode located at said distal end;
at least one electrically conductive element connecting said connector to said electrode;
a tubular lead tip located at said distal end; and
at least a portion of said cardiac pacing element covered with a therapeutic-releasing material having a first biocompatible polymeric material having at least one surface and a therapeutic agent releasably incorporated in at least a portion thereof, wherein a portion of said first biocompatible polymeric material is impermeable to said therapeutic agent and a second biocompatible polymeric material impermeable to said therapeutic agent covering substantially all said at least one surface.

36. The therapeutic-releasing material of claim 35 further comprising at least one opening in said second biocompatible polymeric material.

37. The therapeutic-releasing material of claim 35 wherein said first biocompatible polymeric material comprises a fluoropolymer composition.

38. The therapeutic-releasing material of claim 37 wherein said fluoropolymer is porous polytetrafluoroethylene.

39. The therapeutic-releasing material of claim 35 wherein said second biocompatible polymeric material is a silicone composition.

40. The therapeutic-releasing material of claim 35 wherein said therapeutic agent is dexamethasone sodium phosphate.
